(19) United States
(12) Patent Application Publication
(10) Pub. No.: US 2005/0209306 A1
(43) Pub. Date: Sep. 22, 2005

(54) Crystalline [R-(R*,R*)]-2-(4-fluorophenyI)-beta,delta-dihydroxy-5-(1-methylethyl)-3-phenyl- 4-[(phenylamino)carbonyl]-1H-pyrrole-heptanoic acid calcium salt (2:1)

(76) Inventors: Alexandr Jegorov, Dobra voda (CZ); Jiri Faustman, Opava (CZ)

Correspondence Address: MICHAEL A. STEINBERG, PHD.;IVAX CORPORATION, 4400 BISCAYNE BOULEVARD, MIAMI, FL, 33137, US

(21) Appl. No.: 10/316,822

(22) Filed: Dec. 11, 2002

Related U.S. Application Data
(60) Provisional application No. 60/341,133, filed on Dec. 12, 2001.

(51) Int. Cl. A61K 31/401

(52) U.S. Cl. 514/423; 548/537

(57) ABSTRACT

Novel crystalline forms of atorvastatin calcium designated Fa and Je which are prepared by crystallization from non-aqueous, non-polar solvents at a temperature above 90° C. The crystalline forms are useful as agents for treating hyperlipidemia and hypercholesterolemia.

### CROSS-REFERENCE TO RELATED APPLICATIONS

This application claims the benefit of U.S. Provisional Patent Application No. 60/341,133 filed Dec. 12, 2001, which is incorporated herein by reference.

### FIELD OF THE INVENTION

The invention pertains to the Fa and Je crystalline forms of atorvastatin calcium as well as to processes for their preparation. The novel crystalline forms are useful as inhibitors of the enzyme 3-hydroxy-3-methylglutaryl-coenzyme A reductase (HMG-CoA reductase). Thus, the crystalline compounds of the invention are useful as agents for treating hyperlipidemia and hypercholesterolemia.

### BACKGROUND OF THE INVENTION

The present invention relates to crystalline forms Fa and Je of atorvastatin calcium, i.e., [R—(R*,R*)]-2-(4-fluorophenyl)-β,δ-dihydroxy-5-(1-methylethyl)-3-phenyl-4-[(phenylamino)-carbonyl]-1H-pyrrole-heptanoic acid calcium salt (2:1), also known as atorvastatin calcium, the processes for their production and isolation, to pharmaceutical compositions which include the crystalline forms Fa and Je, and a pharmaceutically acceptable carrier, and to a method of administering a therapeutic amount of the pharmaceutical composition for the treatment of hyperlipidemia and hypercholesterolemia. Atorvastatin is prepared as a calcium salt (2:1) since the calcium salt is desirable for atorvastatin formulations like tablets, capsules, powders and the like for oral administration.

Processes for the preparation of atorvastatin calcium and key intermediates are disclosed in the U.S. Pat. Nos. 4,681,893, 5,003,080; 5,097,045; 5,103,024; 5,124,482; 5,149,837; 5,155,251; 5,216,174; 5,245,047; 5,248,793; 5,273,995, 5,280,126; 5,342,952; and 5,397,792, which are herein incorporated by reference. Atorvastatin calcium can exist as an amorphous form or in several crystalline forms. The possible existence of various crystalline forms or amorphous solids, which may affect the stability, pharmacokinetic profile and bioavailability of various dosage forms, is well known (Byrn S. R., Solid-State Chemistry of Drugs, Academic Press, New York, pages 79-148 (1982)). The various crystalline forms have different properties as the result of different arrangement of molecules in the crystal structure, different density of packing, and/or by different hydrogen-bond network. Accordingly, individual crystalline forms may be thought of as distinct solids having distinct advantageous and/or disadvantageous physical properties compared to other forms.

Amorphous atorvastatin calcium can be prepared according to U.S. Pat. Nos. 6,087,511 and 6,274,740 by dissolving atorvastatin calcium in non-hydroxylic solvent with subsequent removal of the solvent. Alternatively, amorphous atorvastatin calcium can be prepared according to WO 01/42209 by precipitating atorvastatin calcium from a solvent, in which atorvastatin calcium is soluble, by adding a solvent in which atorvastatin calcium is insoluble. Amorphous atorvastatin calcium can also be formed in an aqueous solution, e.g., by the reaction of an atorvastatin sodium salt and a suitable calcium salt at ambient temperature. Since such solutions are difficult to filter, various processes for the preparation of crystalline atorvastatin calcium have been developed.

Crystalline forms I, II, III, and IV of atorvastatin calcium are described in U.S. Pat. Nos. 5,969,156 and 6,121,461 together with their pseudopolymorphic hydrates, which may differ in the content of water in a particular crystalline form. Atorvastatin calcium form V is described in WO 01/36384, and the same denomination is used for forms described in WO 02/057274 and WO 02/057229. Other forms denominated as forms VI, VII, VIII, IX, X, and XI are described in WO 02/43732. Still other forms designated as forms X, A, B1, B2, C, D, and E are described in WO 02/051804. All of these forms are characterized by their distinct X-ray powder diffraction pattern defined as a list of 2θ values obtained with certain source of X-ray radiation, which can be easily calculated for any other source of radiation by the Bragge equation. While the inventors of these patents claim certain processing and therapeutic advantages of their forms, advantages may yet be realized by other heretofore undiscovered forms of atorvastatin calcium.

It has been found that a particular disadvantage of at least some of these forms is either the high surface area of amorphous or semicrystalline forms, and/or high degree of solvatation/hydration leading to the instability of atorvastatin calcium which can result in the formation of atorvastatin degradation products, namely lactone formation, oxidation, elimination of water from its structure, and/or combinations thereof.

### SUMMARY OF THE INVENTION

The present invention provides for new atorvastatin calcium crystalline forms Fa and Je characterized by the X-ray powder diffraction pattern, solid state ¹³C NMR spectra, and differential scanning calorimetry curves.

In another aspect, the present invention provides new processes for preparation of atorvastatin calcium crystalline forms Fa and Je.

In another aspect, the invention provides pharmaceutical compositions and dosage forms comprising atorvastatin calcium crystalline forms Fa and Je.

A still further embodiment of the present invention is a method of treating hyperlipidemia or hypercholesteremia with a pharmaceutical composition containing a therapeutically effective amount of atorvastatin calcium crystalline forms Fa and Je.

### BRIEF DESCRIPTION OF THE DRAWINGS

The invention is further described by the following non-limiting examples, which refer to the accompanying FIGS. 1 to 7, which are briefly described below.

FIG. 1 is a characteristic powder diffraction pattern of atorvastatin calcium crystalline form Fa obtained using CuKα radiation.

FIG. 2 is a comparison of characteristic solid state ¹³C NMR spectra of atorvastatin calcium crystalline form I (top), crystalline form Fa (middle), and crystalline form Je (bottom).

FIG. 3 is a detailed view on solid state ¹³C NMR spectrum of atorvastatin calcium crystalline form Fa.

FIG. 4 is a characteristic differential scanning calorimetry curve of atorvastatin calcium form Fa.

FIG. 5 is a characteristic powder diffraction pattern of atorvastatin calcium crystalline from Je obtained using CuKa radiation.

FIG. 6 is a detailed view on solid state ¹³C NMR spectrum of atorvastatin calcium crystalline form Je.

FIG. 7 is a characteristic differential scanning calorimetry curve of atorvastatin calcium crystalline form Je.

### DETAILED DESCRIPTION OF THE INVENTION

Surprisingly and unexpectedly, it has been discovered that atorvastatin can be prepared in additional crystalline forms. Thus, the present invention provides atorvastatin calcium (2:1) in two new crystalline forms denominated as crystalline form “Fa” and crystalline form “Je”.

Crystalline forms Fa and Je exhibit different physical characteristic compared to the previously described forms based on their X-ray powder patterns, solid state ¹³C NMR and differential scanning calorimetry curves. In addition, the process for synthesizing forms Je and Fa provides an additional advantage in the elimination of polar residual solvents from the final crystalline atorvastatin calcium thereby contributing to the better stability of atorvastatin calcium with respect to possible above mentioned degradation processes.

The patents, published applications, and scientific literature referred to herein establish the knowledge of those with skill in the art and are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001).

As used in this specification, the singular forms “a,” “an” and “the” specifically also encompass the plural forms of the terms to which they refer, unless the content clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the state value by a variance of 20%.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

In the specification and the appended claims, the singular forms include plural referents unless the context clearly dictates otherwise.

Any suitable materials and/or methods known to those of skill can be utilized in carrying out the present invention. However, preferred materials and methods are described. Materials, reagents and the like to which reference is made in the following description and examples are obtainable from commercial sources, unless otherwise noted.

Reference is made hereinafter in detail to specific embodiments of the invention. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention as defined by the appended claims. In the following description, numerous specific details are set forth in order to provide a through understanding of the present invention. The present invention may be practiced without some or all of these specific details. In other instances, well known process operations have not been described in detail, in order not to obscure the present invention.

This invention is related to crystalline forms Fa and Je of [R—(R*,R*)]-2-(4-fluorophenyl)-β,δ-dihydroxy-5-(1-methylethyl)-3-phenyl-4-[(phenylamino)-carbonyl]-1H-pyrrole-heptanoic acid calcium salt (2:1) having the following generic chemical structure:

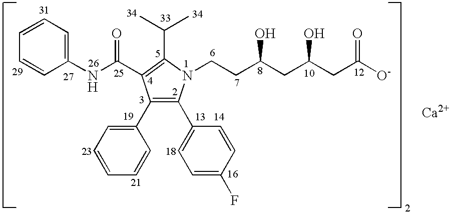

The invention is further directed to the professes for the production and isolation of forms of Fa and Je, to pharmaceutical compositions which include the crystalline forms Fa and Je, and a pharmaceutically acceptable carrier, and to a method of administering a therapeutic amount of the pharmaceutical composition for the treatment of hyperlipidemia and hypercholesterolemia. The Fa and Je crystalline forms of atorvastatin calcium are useful as inhibitors of the enzyme 3-hydroxy-3-methylglutaryl-coenzyme A reductase, and therefore, are useful as agents for treating hyperlipidemia and hypercholesterolemia.

The Fa and Je crystalline forms are characterized by their distinctive X-ray powder diffraction patterns, solid state 1³C nuclear magnetic resonance (NMR) spectra, and differential scanning calorimetry data. These forms are different from other forms known in the prior art.

### Crystalline Form Fa

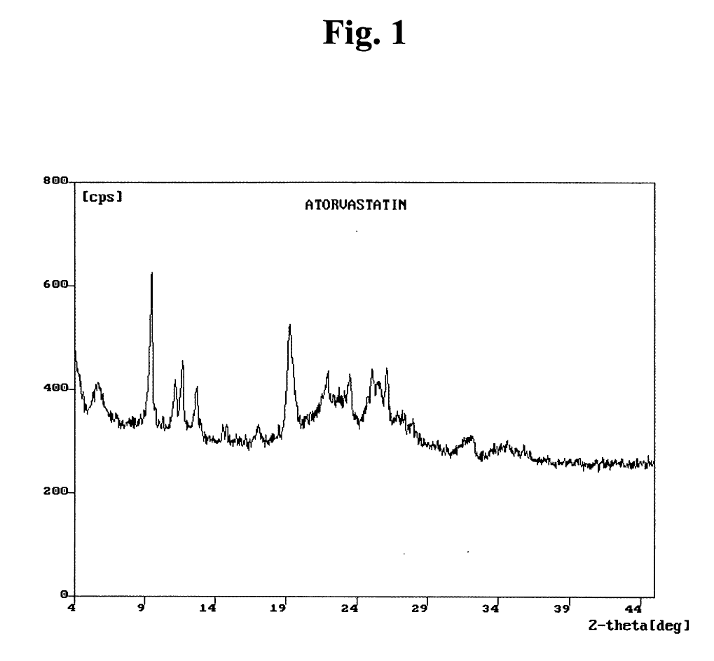

Atorvastatin calcium form Fa is characterized by its X-ray powder diffraction pattern measured on a XRD 3000P diffractometer Seifert with CuKα radiation. Table 1 below lists 2θ values, d-spacings, and roughly relative intensities from the diffractogram illustrated in FIG. 1.

TABLE 1

| 2θ Angle (degrees) | d-Spacings | Relative Intensities |
|---|---|---|
| 10.21 | 10.058 | 725 |
| 11.34 | 9.060 | 712 |
| 11.94 | 8.610 | 374 |
| 12.99 | 7.913 | 169 |
| 18.87 | 5.461 | 1000 |
| 23.14 | 4.462 | 316 |
| 25.04 | 4.129 | 273 |

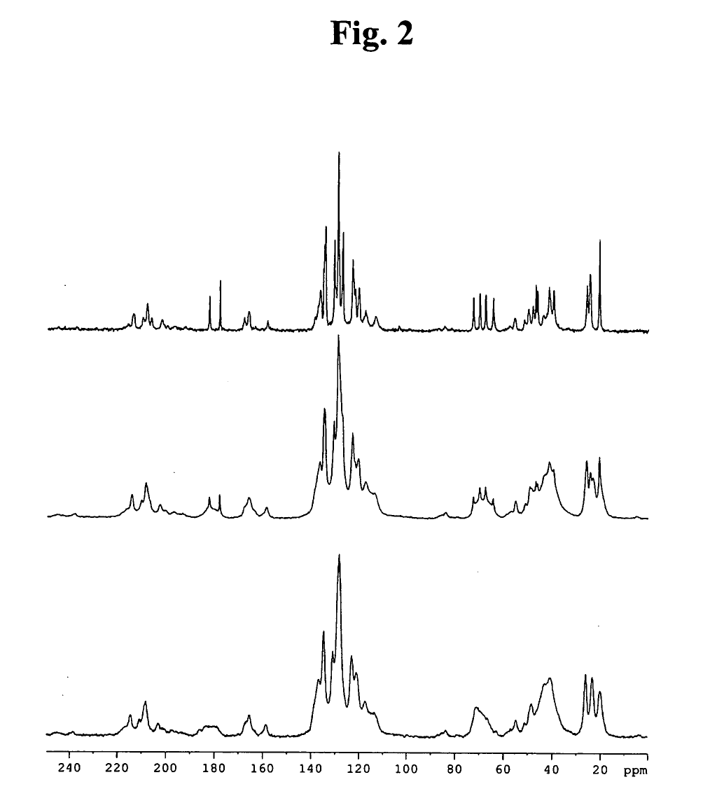

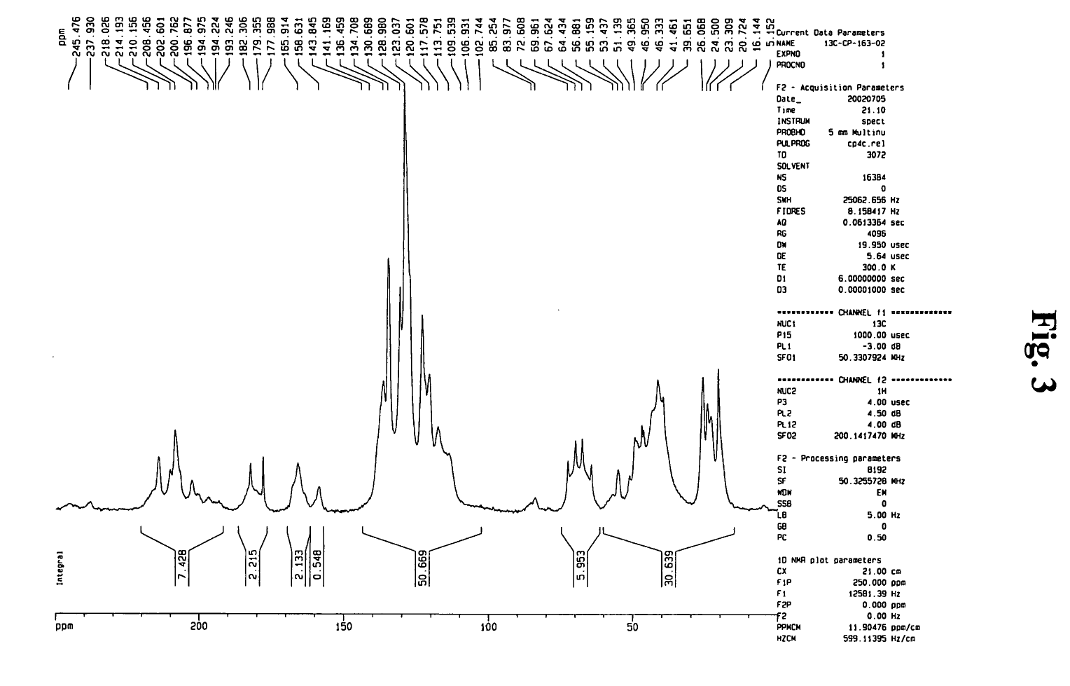

Atorvastatin calcium crystalline form Fa is further characterized by its solid state ¹³C NMR spectrum wherein a chemical shift is expressed as parts per million (ppm) as measured on a Bruker DSX 200 spectrometer (Karlsruhe, Germany) operating at 50.33 MHz and 200.14 MHz for ¹³C and ¹H, respectively. The spectrum for atorvastatin calcium crystalline form Fa is the middle graph in FIG. 2, which shows a comparison of crystalline form Fa with crystalline form I (top) and crystalline form Je (bottom). A detailed view of the spectrum and the values of the chemical shifts is illustrated in FIG. 3. In comparison to other crystalline forms, crystalline form Fa exhibits signals at about 20.7, about 23.3, about 24.5, and about 26.1 ppm. Further in comparison to other forms, crystalline form Fa exhibits multiple peaks in the 30 to 50 ppm region and in the 60 to 75 ppm region with specific signals at about 64.4, about 67.6, about 70.0, and about 72.6 ppm.

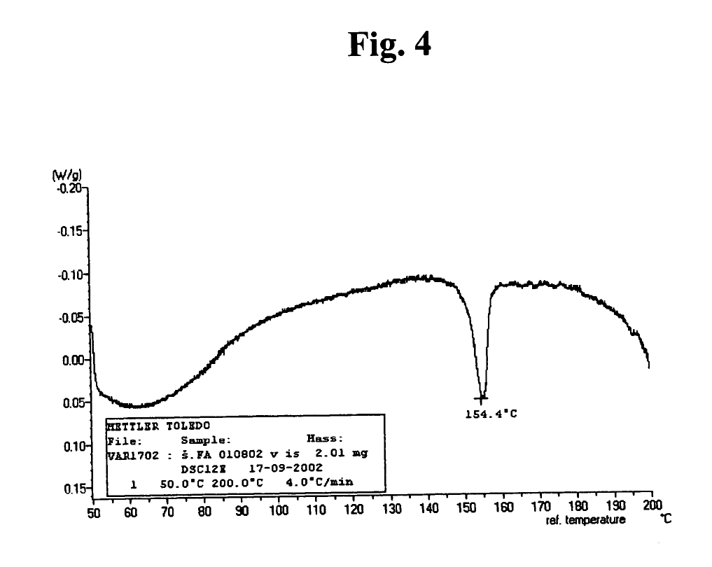

Atorvastatin calcium crystalline form Fa is still further characterized by its differential scanning calorimetry curve. In contrast to other crystalline forms, particularly various solvates and hydrates, no significant activity is present between 100° and 140° C. Crystalline form Fa is characterized by single transition at between 154° C. and 155° C. The calorimetry data is illustrated in FIG. 4.

### Crystalline Form Je

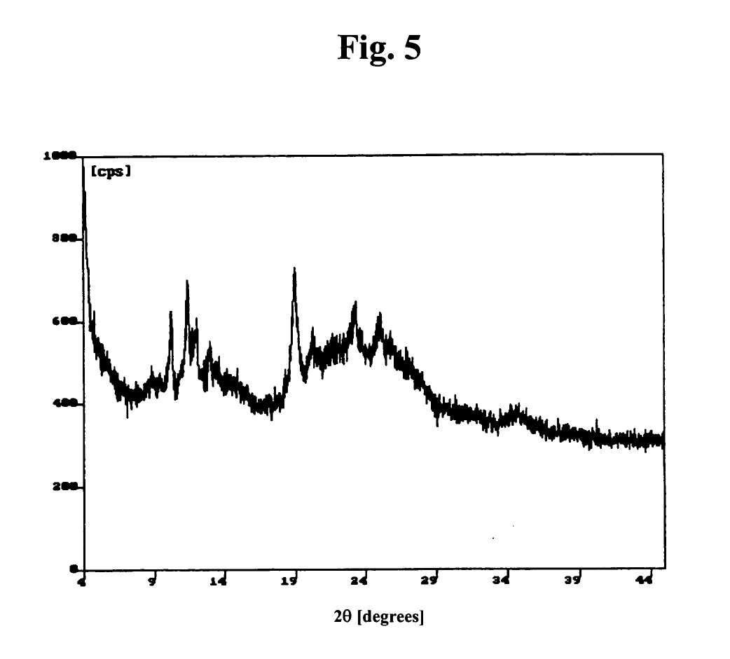

Atorvastatin calcium form Je is characterized by its X-ray powder diffraction pattern measured on a XRD 3000P diffractometer Seifert with CuKα radiation. Table 2 below lists 2θ values, d-spacings, and roughly relative intensities from the diffractogram illustrated in FIG. 5.

TABLE 2

| 2θ Angle (degrees) | d-Spacings | Relative Intensities |
|---|---|---|
| 5.66 | 18.133 | 186 |
| 9.47 | 10.839 | 1000 |
| 11.14 | 9.223 | 328 |
| 11.69 | 8.791 | 460 |
| 12.70 | 8.095 | 306 |
| 19.26 | 5.351 | 680 |
| 21.95 | 4.702 | 310 |
| 22.70 | 4.548 | 200 |
| 23.46 | 4.403 | 299 |
| 25.04 | 4.129 | 356 |
| 26.09 | 3.966 | 392 |

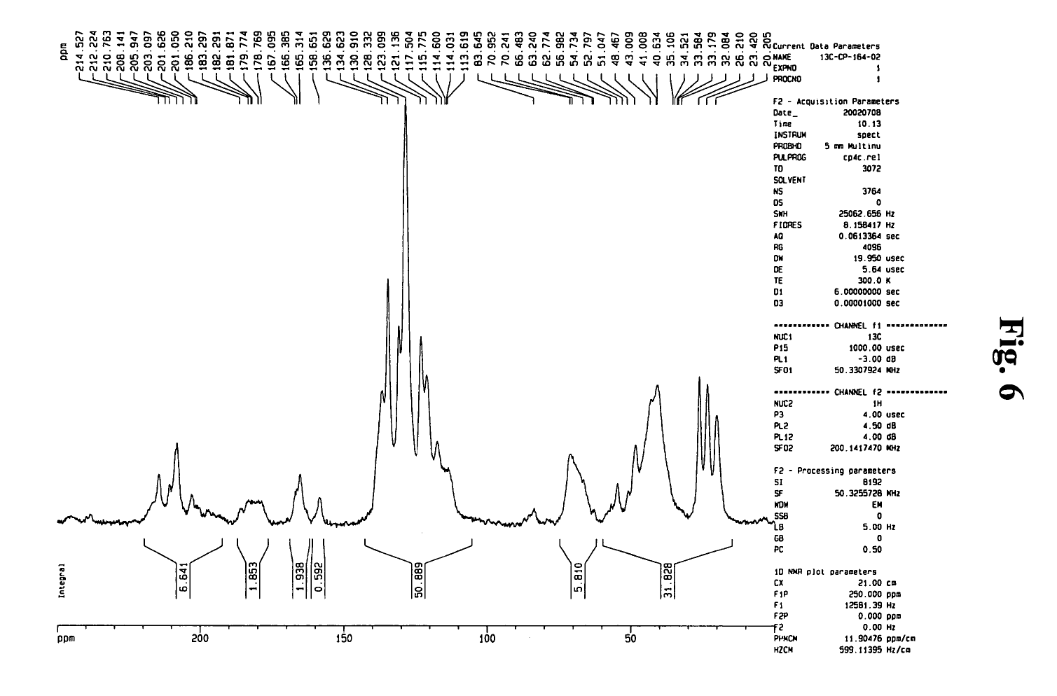

Atorvastatin calcium crystalline form Je is further characterized by its solid state ¹³C NMR spectrum wherein a chemical shift is expressed as parts per million (ppm) as measured on a Bruker DSX200 spectrometer (Karlsruhe, Germany) operating at 50.33 MHz and 220.14 MHz for ¹³C and ¹H, respectively. The spectrum for atorvastatin calcium crystalline form Je is the bottom graph in FIG. 2, which shows a comparison of crystalline form Fa with crystalline form I (top) and crystalline form Fa (middle). A detailed view of the spectrum for crystalline form Je and the values of chemical shifts are illustrated in FIG. 6. In comparison to other forms, form Je exhibits typical signals at about 20.2, about 23.4, and about 26.2 ppm. In comparison to other forms, crystalline form Je exhibits broad shaped peaks in the 30 to 50 ppm region and in the 60 to 75 ppm region as opposed to the fine structure shown for crystalline form I and crystalline form Fa.

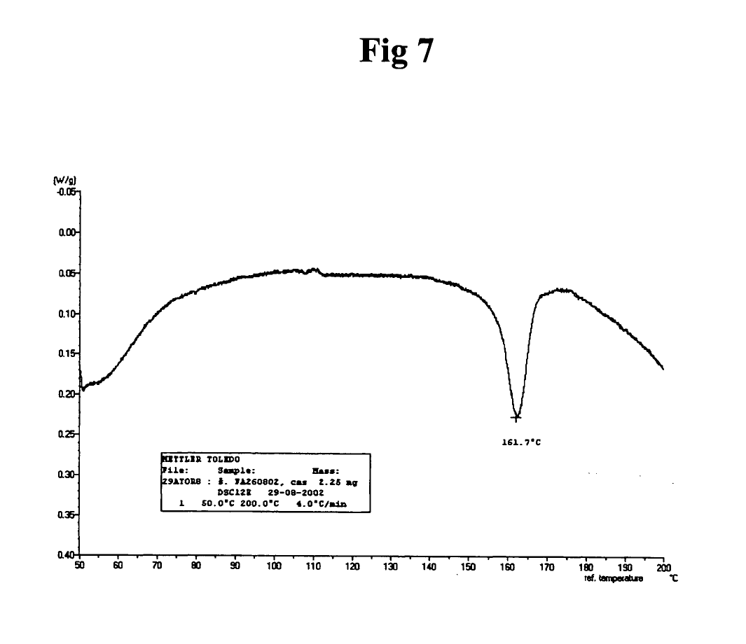

Atorvastatin calcium form Je is still further characterized by its differential scanning calorimetry curve as illustrated in FIG. 7. In contrast to other crystalline forms, particularly various solvates and hydrates, no significant activity is present between 100° and 150° C. The crystalline form Je is characterized by single transition temperature between 162° and 163° C.

With respect to differential scanning calorimetry (DSC), because of the natural variation between independent samples, the manner of sample preparation, particle size and their possible micronization; the positions of diffraction lines, particularly their intensities, can slightly differ for individual samples. In addition, the position of maximum at the DSC curve can be slightly affected by the temperature program used.

Method of Preparing Crystalline Forms Fa and Je

The present invention also provides for a method for the preparation of crystalline forms Fa and Je of atorvastatin calcium (2:1). The method comprises exposing atorvastatin to temperature conditions, which yield crystalline forms Fa or Je. The precise conditions under which forms Fa and Je are formed may be empirically determined.

Crystalline atorvastatin calcium forms Fa and Je may be prepared by crystallization under controlled conditions. In particular, they can be prepared by crystallization from non-aqueous, non-polar solvents at a temperature above 90° C. Suitable non-aqueous, non-polar solvents include, but are not limited to, hydrocarbons, e.g., octane, heptane, isooctane, methylcyclohexane or the like, and mixtures thereof.

In one embodiment, a slurry of an amorphous atorvastatin is formed in aqueous media by precipitation of a atorvastatin soluble salt such as an atorvastatin alkali salt with a suitable calcium salt such as calcium acetate. The slurry is directly mixed with the non-aqueous, non-polar solvent. Crystallization is then carried out at a temperature above 90° C.

In another embodiment, amorphous atorvastatin calcium is suspended in water and hydrated. The water was replaced with a non-aqueous solvent and the solution is subjected to azeotropic distillation at a temperature above 90° C. to form the crystallized form of atorvastatin calcium of the present invention. In still another embodiment, atorvastatin calcium crystalline form I is suspended in a non-aqueous solvent and the resultant solution is heated to a temperature above 90° C. to cause crystallization and the formation of atorvastatin calcium crystalline form of the present invention.

The elevated temperature for crystallization is preferably above 90° C., and most preferably between 90° and 120° C. Such temperatures can be achieved also with solvents having low boiling point such as hexane under elevated pressure. In particular, the process can be used in order to obtain atorvastatin calcium crystalline forms Fa and Je with a defined amount of water and with defined size of particles, which can be easily isolated by filtration. Under controlled conditions, pure atorvastatin calcium crystalline form Fa can be isolated. Such conditions include the use of a lower temperature for crystallization, shorter times of crystallization, and preferably the use of methylcyclohexane or isooctane. By increasing the temperature, the atorvastatin calcium crystalline form Fa becomes metastable and re-crystallizes into atorvastatin calcium form Je. Such transition is characterized by the shift of first two intensive diffraction lines in the X-ray powder patterns and by characteristic changes in the region 15-30 ppm in the ¹³C NMR spectra. These changes can be monitored. However, this transition can be more easily monitored by DSC. The transition from crystalline form Fa to crystalline form Je is accompanied by the decreasing of intensity of the peak at about 155° C., increasing of intensity of the peak at about 163° C., and finally the disappearance of this doublet and formation of a single peak at about 163° C. The formation of the single peak at about 163° C. can be used as the end point for the monitoring of transition of atorvastatin calcium crystalline form Fa to crystalline form Je.

Therefore, under controlled conditions, pure atorvastatin calcium crystalline form Je can be isolated. Such conditions include the use of higher temperature, longer time of crystallization, and/or the use of heptane or octane as solvents as compared to the process for making crystalline form Fa. The isolated crystals may be dried by conventional means. Advantageously, the process is carried out in an inert atmosphere under the inert gas, e.g., nitrogen, argon or the like.

The methods of the present invention are intended for use with any subject that may experience the benefits of the methods of the invention. Thus, in accordance with the invention, “subjects” include humans as well as non-human subject, particularly domesticated animals.

It will be understood that the subject to which a compound of the invention is administered need not suffer from a specific traumatic state. Indeed, the compounds of the invention may be administered prophylactically, prior to any development of symptoms. The term “therapeutic,” “therapeutically,” and permutations of these terms are used to encompass therapeutic, palliative as well as prophylactic uses. Hence, as used herein, by “treating or alleviating the symptoms” is meant reducing, preventing, and/or reversing the symptoms of the individual to which a compound of the invention has been administered, as compared to the symptoms of an individual receiving no such administration.

The term “therapeutically effective amount” is used to denote treatments at dosages effective to achieve the therapeutic result sought. Furthermore, one of skill will appreciate that the therapeutically effective amount of the compound of the invention may be lowered or increased by fine tuning and/or by administering more than one compound of the invention, or by administering a compound of the invention with another compound. The invention therefore provides a method to tailor the administration/treatment to the particular exigencies specific to a given mammal. As illustrated in the following examples, therapeutically effective amounts may be easily determined for example empirically by starting at relatively low amounts and by step-wise increments with concurrent evaluation of beneficial effect.

The compounds according to the invention are optionally formulated in a pharmaceutically acceptable vehicle with any of the well known pharmaceutically acceptable carriers, including diluents and excipients (see Remington's Pharmaceutical Sciences, 18^{th} Ed., Gennaro, Mack Publishing Co., Easton, Pa. 1990 and Remington: The Science and Practice of Pharmacy, Lippincott, Williams & Wilkins, 1995). While the type of pharmaceutically acceptable carrier/vehicle employed in generating the compositions of the invention will vary depending upon the mode of administration of the composition to a mammal, generally pharmaceutically acceptable carriers are physiologically inert and non-toxic. Formulations of compositions according to the invention may contain more than one type of compound of the invention), as well any other pharmacologically active ingredient useful for the treatment of the symptom/condition being treated.

The crystalline compounds of the present invention can be prepared into a pharmaceutical composition by admixing the compound with a pharmaceutically acceptable carrier, adjuvant or vehicle. The resultant pharmaceutical composition can be administered in a wide variety of dosage forms, e.g., oral, topical, parenteral or the like. It will be obvious to those skilled in the art that such dosage forms, e.g., powders, tablets, pills, capsules, aggregates, suppositories, granules and the like, or liquid forms, e.g., solutions, suspensions, or emulsions may comprise as the active component of the present invention. In solid dosage form, the atorvastatin calcium crystalline form Fa or Je is finely divided or mixed with one or more inactive ingredients, which can act as inactive filling materials, taste or flavor corrigenda, chemical preservatives, solubilizers, lubricants, and the like. In liquid form, the atorvastatin calcium crystalline form Fa or Je is suspended, emulsified or dissolved in suitable vehicles containing various inactive components, e.g., solvents, buffers, stabilizers, colorants, flavors, and the like. The preferred unit dosages of the pharmaceutical composition of this invention typically contain from 0.5 to 100 mg of atorvastatin calcium crystalline form Fa or Je, or a mixture of crystalline forms Fa and Je.

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### EXAMPLES

The following examples are intended to further illustrate certain preferred embodiments of the invention and are not limiting in nature. Those skilled in the art will recognize, or be able to ascertain, using no more than routine experimentation, numerous equivalents to the specific substances and procedures described herein.

### Example 1

Amorphous atorvastatin calcium (20 g) was suspended in water (160 ml) and hydrated 20 minute at 100° C. Water was replaced with isooctane (160 ml) and crystallization was carried out for 6 hours at 99° C. After cooling to a temperature between 20° and 30° C., the mixture was filtered, and the resultant solid was dried at 100° C. for 30 hours at atmospheric pressure and under a nitrogen stream to give atorvastatin calcium crystalline form Fa (17.3 g).

### Example 2

Atorvastatin calcium crystalline form I (10 g) was suspended in n-octane (200 ml) and the mixture was heated with stirring to 120° C. for 30 minutes under the stream of nitrogen. After cooling to a temperature between 20° and 30° C., the mixture was filtered, washed with petroleum ether and the resultant solid was dried at 50° C. under vacuum for 2 hours to give atorvastatin calcium crystalline form Je (9.1 g).

### Example 3

A semicrystalline slurry of atorvastatin calcium was obtained by the reaction of 10 g of atorvastatin sodium salt and an equimolar amount of calcium acetate in a 5% aqueous methanol solution. The slurry was filtered, washed with aqueous methanol and the solvent was replaced by n-octane (250 ml). The mixture was heated with stirring to 120° C. for 30 minutes under the stream of nitrogen with azeotropic distillation of water. After cooling to a temperature between 20° and 30° C., the mixture was filtered, washed with petroleum ether and the remaining solid was dried at 50° C. under vacuum for 2 hours to give atorvastatin calcium crystalline form Je (7.7 g).

### Example 4

Amorphous atorvastatin calcium (20 g) was suspended in water (160 ml) and hydrated 20 min at 100° C. The hydrated atorvastatin calcium was dried for 2 hours at 100° C. The dried atorvastatin calcium was re-supplemented with water (13 g) followed by the addition of n-heptane (160 ml). The mixture was subjected to azeotropic distillation for 6 hours at a final temperature of 98.5° C. Monitoring of the reaction by DSC revealed that atorvastatin calcium crystalline form Fa was formed in pure state within one hour. After two hours, a mixture of atorvastatin calcium crystalline forms Fa and Ja (roughly 1:1) was formed. After 3 hours, the product was almost entirely the atorvastatin calcium crystalline form Je. After cooling to a temperature between 20° and 30° C., the mixture was filtered, and the remaining solid was dried at 100° C. for 30 hours at atmospheric pressure and under nitrogen stream to give atorvastatin calcium crystalline form Je (18.3 g).

While the salient features have been illustrated and described with respect to particular embodiments, it should be readily apparent that modifications can be made within the spirit and scope of the invention, and it is therefore not desired to limit the invention to the exact details shown and described.

1. A compound comprising atorvastatin calcium crystalline form Fa or a pseudopolymorph thereof.

2. The compound of claim 1, having an X-ray powder diffraction pattern having at least one of the following 20 values measured using CuKa radiation: about 10.2, about 11.3, or about 18.9.

3. The compound of claim 1, having a solid state ¹³C NMR spectrum having at least one chemical shift at about 20.7, about 23.3, about 24.5, or about 26.1 ppm.

4. The compound of claim 3, wherein the solid state ¹³C NMR spectrum further includes at least one chemical shifts at about 64.4, about 67.6, about 70.0, and about 72.6 ppm.

5. The compound of claim 1, wherein a differential scanning calorimetry curve of said compound includes a single transition between 154° C. and 155° C.

6. A compound comprising atorvastatin calcium crystalline form Je or a pseudopolymorph thereof.

7. The compound of claim 6, having an X-ray powder diffraction pattern having at least one of the following 20 values measured using CuKa radiation: about 9.5, about 11.1, about 11.7, about 12.7, and about 19.3.

8. The compound of claim 6, having a solid state ¹³C NMR spectrum having at least one chemical shift at about 20.2, about 23.4, and about 26.2 ppm.

9. The compound of claim 6, wherein a differential scanning calorimetry curve of said compound includes a single transition between 162° C. and 163° C.

10. A process for preparing atorvastatin calcium crystalline forms Fa and Je or pseudoplymorphs thereof, said process comprising the steps of suspending an aqueous slurry of atorvastatin calcium salt in a non-aqueous, non-polar solvent and heating the mixture above 90° C.

11. A process for preparing of atorvastatin calcium crystalline forms Fa and Je or pseudoplymorphs thereof, said process comprising the steps of:
a) hydration of amorphous atorvastatin calcium;
b) suspending of hydrated atorvastatin calcium in a non-aqueous, non-polar solvent;
c) heating to a temperature and for a period of time sufficient to form atorvastatin calcium crystalline form Fa or Je, or a mixture of crystalline forms Fa and Je; and
d) recovering said atorvastatin calcium crystalline forms from the suspension.

12. A process according the claim 11, where the hydration of amorphous atorvastatin calcium is carried out at a temperature above 90° C.

13. A process for preparing atorvastatin calcium crystalline forms Fa or Je, said process comprising the steps of:
a) suspending atorvastatin calcium in a non-aqueous, non-polar solvent;
b) heating to a temperature and for a period of time sufficient to form atorvastatin calcium crystalline forms Fa or Je, or a mixture of crystalline forms Fa and Je; and
c) recovering said atorvastatin calcium crystallized forms from the suspension.

14. A process for purification of atorvastatin calcium crystalline forms Fa and Je by removing of residual solvents, water, or other impurities from atorvastatin calcium by distillation or azeotropic distillation of these solvents from atorvastatin calcium suspended in non-aqueous solvents.

15. The process as in any one of claims 10, 11, 13 or 14, in which the non-aqueous, non-polar solvent is at least one hydrocarbon.

16. The process as in any one of claims 10, 11, 13 or 14, in which the non-aqueous, non-polar solvent is selected from the group consisting of hexane, heptane, octane, isooctane, cyclohexane, methylcyclohexane, and mixtures thereof.

17. A pharmaceutical composition comprising the compound according to claim 1 or 6 in an admixture with a pharmaceutically acceptable carrier, adjuvant or vehicle.

18. A method of treating hyperlipidemia and hypercholesterolemia by administering a therapeutically effective amount of the compound according to claim 1 or 6.
